(19) United States
(12) Patent Application Publication
(10) Pub. No.: US 2026/0344992 A1
(43) Pub. Date: Nov. 5, 2026

(54) DEVICES, SYSTEMS, AND METHODS FOR COOLING A ROBOTIC CAMERA

(71) Applicant: Vicarious Surgical Inc., Waltham, MA (US)

(72) Inventors: Matthew Kincaid, Medford, MA (US); Eric Kline, Malden, MA (US); Evan Stypinski, Saint Petersburg, FL (US); Paul Birkner, Framingham, MA (US)

(21) Appl. No.: 19/125,457

(22) PCT Filed: Oct. 31, 2023
(86) PCT No.: PCT/US2023/036347

§ 371 Date: Apr. 29, 2025

Related U.S. Application Data
(60) Provisional application No. 63/421,028, filed on Oct. 31, 2022.

(51) Int. Cl. A61B 1/12; A61B 1/00

(52) U.S. Cl. CPC A61B 1/128; A61B 1/00009; A61B 1/00149

(57) ABSTRACT

Systems and methods for cooling a robotic camera including a housing including at least one camera module, electronic components, and a hollow chamber. A fluidic circuit in fluid communication with the hollow chamber and a pump removing heat from the housing.

### RELATED APPLICATION

This application claims the benefit of the filing date, under 35 U.S.C. § 119 (e), of U.S. Provisional Application No. 63/421,028, filed on Oct. 31, 2022, the entire contents of which are incorporated herein by reference.

### BACKGROUND OF THE DISCLOSURE

Surgical robotic systems permit a surgeon (also described herein as an “operator” or a “user”) to perform an operation using robotically-controlled instruments to perform tasks and functions during a procedure. The surgeon may use a visualization system to view or watch the operation, including to view images from a camera or cameras showing the patient and/or mounted to the robotically-controlled instruments.

The camera systems used in the surgical robotic system may contain a variety of electrical components installed within a housing that emit heat when in use, including the camera itself, light sources (such as light emitting diodes (LEDs)), power supplies, processors, and other components. These features may cause the temperature of the camera system and housing to increase.

Safety standards establish maximum temperatures at which a surgical system component that may come in contact with tissue of the patient can operate in view of the expected length of exposure.

Additionally, the size of the camera housing is preferably of a small size so that the camera system can be inserted through a trocar for laparoscopic surgery and interfere with maneuvering of surgical robotic tools is reduced.

### SUMMARY

The present disclosure is directed to a robotic surgical system including a housing including at least one camera module, one or more electronic components, and a hollow chamber proximate to the one or more electronic components. The robotic surgical system may also include a camera support assembly supporting the housing and including an arm with at least a first joint. The robotic surgical system may also include a pump and a fluidic circuit in fluid communication with the hollow chamber and the pump and extending along at least part of the camera support assembly.

In some embodiments the hollow chamber is formed along an interior wall of the housing opposite the at least one camera module and includes an ingress aperture and an egress aperture. In some embodiments, the robotic surgical system further includes a first plate in the housing forming a wall of the hollow chamber. In some embodiments, the hollow chamber includes a plurality of fluid channels therein. In some embodiments, the robotic surgical system further includes a thermally-conductive material, such as a thermally-conductive putty, contacting at least a portion of the at least one camera module, the one or more electronic components, and a wall of the hollow chamber.

In some embodiments, the fluidic circuit includes a first portion having a first inner diameter and a first outer diameter and a second portion having a second inner diameter and a second outer diameter. In some embodiments, the second inner diameter is greater than the first inner diameter. In some embodiments, the first inner diameter is between 0.1 mm and 0.4 mm and the first outer diameter is between 0.5 mm and 1 mm. In some embodiments, the first inner diameter is about 0.25 mm and the first outer diameter is about 0.75 mm. In some embodiments, the second inner diameter is between 0.5 mm and 1 mm. In some embodiments, the second inner diameter is about 0.75 mm. In some embodiments, the second inner diameter is approximately equal to the first outer diameter.

In some embodiments, at least the first portion of the fluidic circuit is a kink-resistant polymer tubing. In some embodiments, the first portion and the second portion of the fluidic circuit including a polymer independently selected from a group consisting of polyvinyl chloride, silicone, polyethylene, polyamide, polyurethane, and combinations thereof. In some embodiments, first portion and the second portion of the fluidic circuit comprise medical grade polyvinyl chloride. In some embodiments, at least the first portion of the fluidic circuit comprises a polymer material with a Durometer Shore A hardness rating of about 80. In some embodiments, at least the second portion of the fluidic circuit comprises a polymer material with a Durometer Shore A hardness rating of about 65. In some embodiments, the tubing may be biocompatible. In some embodiments the tubing may have a smooth inner bore. In some embodiments, the tubing may be soft, flexible, and kink resistant. In some embodiments, the tubing may be capable of being sterilized, e.g., by gas, autoclave, or radiation. Portions of the tubing may be capable of being bonded directly to other portions of the tubing using, for example, ultraviolet light curable glue or cyclohexane solvent.

In some embodiments, the robotic surgical system further includes a fill port and a purge valve in fluid communication with the fluidic circuit. In some embodiments, the robotic surgical system further includes an external reservoir in fluid communication with the fluidic circuit. In some embodiments, the fluidic circuit comprises a first coil of tubing proximate the first joint. In some embodiments, the camera support additionally includes a second joint and the fluidic circuit comprises a second coil of tubing proximate the second joint. In some embodiments, the first coil of tubing comprises a number of coils sufficient to permit 540 degrees of rotation by the first joint.

In some embodiments, the pump is a peristaltic pump. In some embodiments, the pump is configured to be driven by a pump motor. In some embodiments, the pump comprises a plurality of sets of rollers. In some embodiments, the robotic surgical system has an internal portion and an external portion, the internal portion including the housing and the camera support and being configured to be placed at least partially inside a patient during a surgical procedure, and the external portion configured to be outside a patient during a surgical procedure. In some embodiments, the pump is in the external portion.

In some embodiments, the robotic surgical system further includes an external reservoir in fluid communication with the fluidic circuit. In some embodiments, the housing and the camera support form at least part of a laparoscopic camera.

The present disclosure is directed to a method for cooling a robotic surgical system that includes (i) providing a robotic surgical system including a housing with at least one camera module, one or more electronic components, and a hollow chamber proximate to the one or more electronic components; and (ii) pumping a fluid from an external reservoir through a fluidic circuit including the hollow chamber.

In some embodiments, the method also includes removing heat from the hollow chamber of the housing by means of the fluid. In some embodiments, the method also includes removing heat from the hollow chamber removes sufficient heat to maintain a temperature at or below about 43 °C on an exterior surface of the electronic housing proximate the hollow chamber. In some embodiments, pumping a fluid through a fluidic circuit including the hollow chamber and an external reservoir comprises establishing a fluid flow sufficient to maintain a temperature within the housing of about 70 °C or less. In some embodiments, pumping a fluid through a fluidic circuit including the hollow chamber and an external reservoir comprises establishing a fluid flow of about 5 mL/minute or more. In some embodiments, pumping a fluid through a fluidic circuit including the hollow chamber and an external reservoir comprises pumping at a pressure of 30 psi or greater. In some embodiments, the method also includes permitting a portion of the fluid to cool passively in the reservoir. In some embodiments, the method also includes actively cooling a portion of the fluid using a heat exchanger. In some embodiments, the method also includes rotating a joint of the robotic surgical system while permitting coiling or uncoiling of a tube coil proximate to the joint. In some embodiments, the fluid is a liquid. In some embodiments, the fluid is a liquid selected from distilled water or saline. In some embodiments, the fluid is a non-liquid fluid, such as a gas or supercritical fluid.

The present disclosure is directed to a minimally invasive surgical camera including a housing; one or more cameras accommodated within the housing; one or more circuit board assemblies provided within the housing; a hollow cavity in proximity to the one or more circuit board assemblies. The hollow cavity may include a fluid ingress port, a fluid egress port and an interior surface defined by the housing. In some embodiments, the minimally invasive surgical camera further includes a first plate within the housing forming a wall of the hollow cavity. In some embodiments, the fluid ingress port and the fluid egress port are configured to receive a polymer tube. In some embodiments, the hollow cavity comprises a plurality of fluid channels. In some embodiments, the plurality of fluid channels are formed by machining channels in the interior surface, the surface of the first plate, or both.

In some embodiments, the minimally invasive surgical camera further includes a thermally-conductive material, such as a thermally-conductive putty, within the housing and contacting at least a portion of the one or more cameras, the one or more circuit board assemblies, and the hollow cavity. In some embodiments, the hollow cavity is configured to receive a fluid flow at the fluid ingress port and to release the fluid flow at the fluid egress port. In some embodiments, the fluid flow in the hollow cavity receives heat emitted by the one or more cameras, the one or more circuit board assemblies, or both. In some embodiments, the fluid flow removes heat from the hollow cavity.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6 is a schematic depicting a camera and cooling system in accordance with some embodiments.

FIG. 7A is a perspective view of a camera and cooling system in accordance with some embodiments.

FIG. 7B is a front view of a camera housing, camera support assembly, and roll joint in accordance with some embodiments.

FIG. 8 is a rearview cross section of a camera system in accordance with some embodiments.

FIG. 9A is a perspective view of a portion of a camera housing in accordance with some embodiments.

FIG. 9B is a perspective view showing components within a camera housing in accordance with some embodiments.

FIG. 10A is a cross sectional view of a camera housing illustrating a location of a chamber for cooling the camera system relative to the camera housing in accordance with some embodiments.

FIG. 10B is a perspective viewing showing components within a camera housing in accordance with some embodiments.

FIG. 11 is a perspective view of a camera system in accordance with some embodiments with a portion of the housing removed to show components in the interior including a chamber for cooling the camera system.

FIG. 12 is a cross sectional view of a camera housing in accordance with some embodiments.

FIG. 13 is a perspective view of a camera housing, support assembly, and roll joint in accordance with some embodiments.

FIG. 14 is a rear cross sectional view of a camera housing in accordance with some embodiments.

FIG. 15 is a rear cross sectional view of a camera housing, camera support assembly, and roll axis joint in accordance with some embodiments.

FIG. 16 is a schematic depicting a camera and cooling system in accordance with some embodiments.

FIG. 17A is a cross sectional view of a camera housing in accordance with some embodiments taken along plane A as shown in FIG. 17B.

FIG. 17B is a front view of a camera housing in accordance with some embodiments showing plane A.

FIG. 18 is a perspective view of a portion of a camera housing with a hollow chamber including channels in accordance with some embodiments.

FIG. 19 is a perspective view of a portion of a camera housing with a hollow chamber including channels in accordance with some embodiments.

### DETAILED DESCRIPTION

While various embodiments of devices, systems, and methods for cooling a robotic camera are illustrated and described herein, it will be clear to those skilled in the art that such embodiments are provided by way of example. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some exemplary embodiments may be described herein or in documents incorporated by reference as employing a plurality of units to perform exemplary processes, it is understood that exemplary processes may also be performed by one or a plurality of modules. Additionally, it is understood that the term controller/control unit may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or control units or multiple different types of controllers or control units may be employed in performing one or more processes. In some embodiments, different controllers or control units may be implemented in different portions of a surgical robotic systems.

Embodiments taught herein provide systems and methods for cooling a robotic surgical camera, such as a robotic laparoscopic camera. In some embodiments, a fluid flow is provided through a hollow chamber of a camera housing in order to cool the camera housing. The fluid flow may be established by a fluidic circuit including the hollow chamber and an external reservoir of the camera and cooling system.

Advantages of embodiments employing the cooling systems disclosed herein include the ability to maintain a temperature within the camera housing that avoids overheating of the electrical components and the ability to maintain a safe temperature of the external surfaces of the camera housing such that the camera housing may contact tissue of the patient without causing injury. Additionally, embodiments may provide sufficient cooling to avoid or reduce a need for other features that may alternatively be used for cooling, such as fins used to increase surface area and provide cooling. Avoiding or reducing such features may support a smaller camera housing. Additionally, avoiding or reducing such features may improve patient safety, by improving ability to sanitize the camera housing and reducing likelihood of the camera catching on a portion of tissue. In a robotic camera or a camera support assembly including an air gap or bushing, embodiments taught herein may overcome or mitigate an insulating effect of the air gap or bushing, wherein the air gap or bushing reduce the rate of heat transfer away from the camera housing along the camera support assembly.

Some embodiments disclosed herein are implemented on, employ, or are incorporated into a surgical robotic system that includes a camera assembly having at least three articulating degrees of freedom and two or more robotic arms each having at least six articulating degrees of freedom and an additional degree of freedom corresponding to the movement of an associated end-effector (e.g., grasper, manipulator, and the like). In some embodiments, the camera assembly when mounted within a subject (e.g., a patient) can be moved or rotated in a pitch or yaw direction about 180 degrees such that the camera assembly can view rearwardly back towards the insertion site. As such, the camera assembly and the robotic arms can view and operate dexterously forward (e.g., away from the insertion site), to each side, in an upward or downward direction, as well as in the rearward direction to view backwards towards the insertion site. The robot arms and the camera assembly can also move in the roll, pitch and yaw directions.

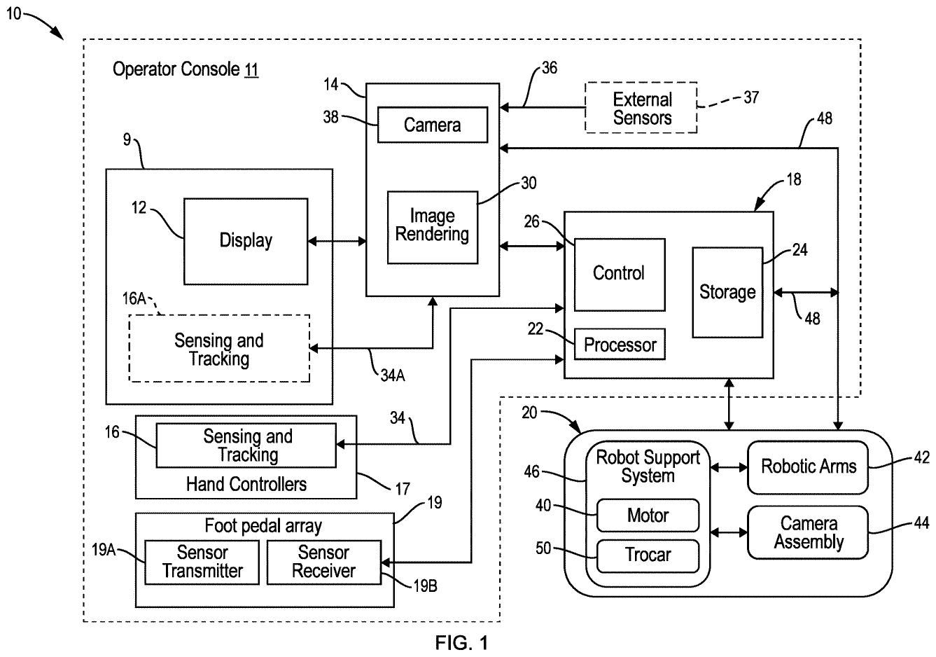

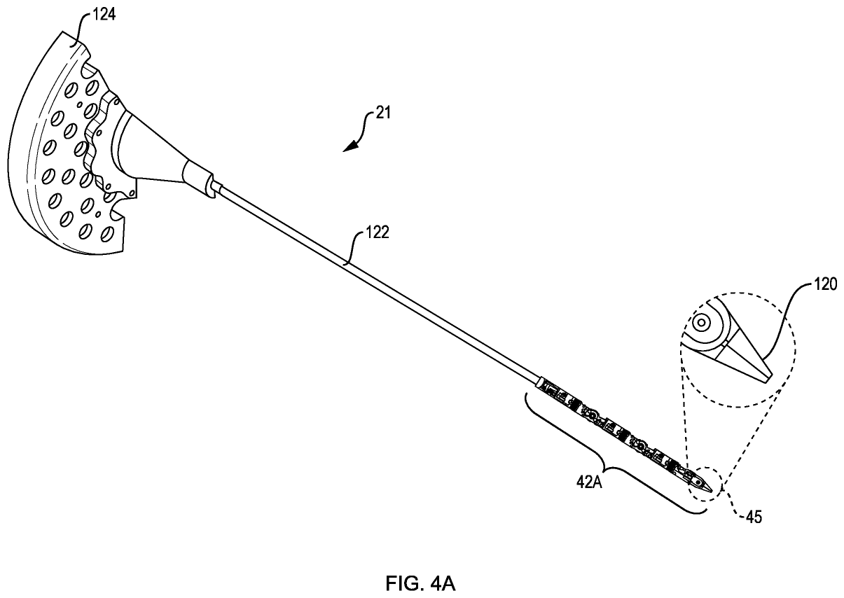

Prior to providing additional specific description of the camera cooling with respect to FIGS. 5-19 a surgical robotic system in which some embodiments could be employed is described below with respect to FIGS. 1-5.

### Surgical Robotic Systems

Some embodiments may be employed with a surgical robotic system. A system for robotic surgery may include a robotic subsystem. The robotic subsystem includes at least a portion, which may also be referred to herein as a robotic assembly that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic unit, a surgical robotic module or a robotic assembly herein. The surgical robotic unit or surgical robotic module can include multiple different subunits or parts that may be inserted into the trocar separately. The surgical robotic unit, surgical robotic module or robotic assembly can include multiple separate robotic arms that are deployable within the patient along different or separate axes. These multiple separate robotic arms may be collectively referred to as a robotic arm assembly herein. Further, a surgical camera assembly can also be deployed along a separate axis. The surgical robotic unit, surgical robotic module, or robotic assembly may also include the surgical camera assembly. Thus, the surgical robotic unit, surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a visualization system 9 with a display device 12, an image computer 14, which may be a three-dimensional (3D) computer, hand controllers 17 having a sensor and tracker 16, and a computer 18. Additionally, the operator console 11 may include a foot pedal array 19 including a plurality of pedals. The foot pedal array 19 may include a sensor transmitter 19A and a sensor receiver 19B to sense presence of a user's foot proximate foot pedal array 19.

The display 12 may be any selected type of display for displaying information, images or video generated by the image computer 14, the computer 18, and/or the robotic subsystem 20. The visualization system 9 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The visualization system 9 can also include an optional sensor and tracker 16A. In some embodiments, the display 12 can include an image display for outputting an image from a camera assembly 44 of the robotic subsystem 20. Discussed in more detail below is a fluid cooled camera assembly 244 suitable for use in place of the camera assembly 44.

In some embodiments, if the visualization system 9 includes an HMD device, an AR device that senses head position, or another device that employs an associated sensor and tracker 16A, the HMD device or head tracking device generates tracking and position data 34A that is received and processed by image computer 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensor and tracker 16A to provide position and orientation tracking of the operator's head. The sensor and tracker 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensor and tracker 16A, can track the position and orientation of the operator's head, generate tracking and position data 34A, and then relay the tracking and position data 34A to the image computer 14 and/or the computer 18 either directly or via the image computer 14.

The hand controllers 17 are configured to sense a movement of the operator's hands and/or arms to manipulate the surgical robotic system 10. The hand controllers 17 can include the sensor and tracker 16, circuitry, and/or other hardware. The sensor and tracker 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensor and tracker 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracker 16A as described above. In some embodiments, the sensor and tracker 16 can be external and coupled to the hand controllers 17 via electricity components and/or mounting hardware. In some embodiments, the optional sensor and tracker 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensor and tracker 16 can employ sensors coupled to the torso of the operator or any other body part. In some embodiments, the sensor and tracker 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensor and tracker 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computer 18 and hence employed by the surgical robotic system 10.

The sensors generate position and/or orientation data indicative of the position and/or orientation of the operator's hands and/or arms. The sensor and tracker 16 16 and/or 16A can be utilized to control movement (e.g., changing a position and/or an orientation) of the camera assembly 44 and robotic arms 42 of the robotic subsystem 20. The tracking and position data 34 generated by the sensor and tracker 16 can be conveyed to the computer 18 for processing by at least one processor 22.

The computer 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computer 18. The motor 40 can thus generate signals for controlling one or more motors that in turn can control and drive the robotic arms 42, including for example the position and orientation of each articulating joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensor and tracker 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure regarding control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes a HMD, the display can include the built-in sensor and tracker 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracker. In some embodiments, the sensor and tracker 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensor and tracker 16A for tracking at least a portion of the operator's head.

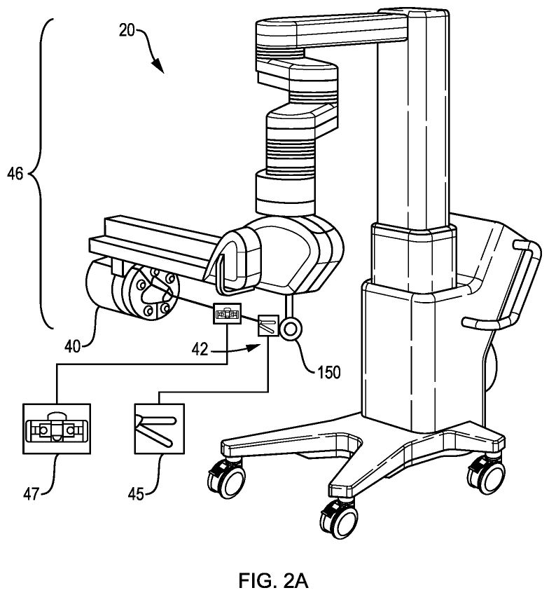

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes the display 12, the hand controllers 17, and also includes one or more additional controllers, such as the foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

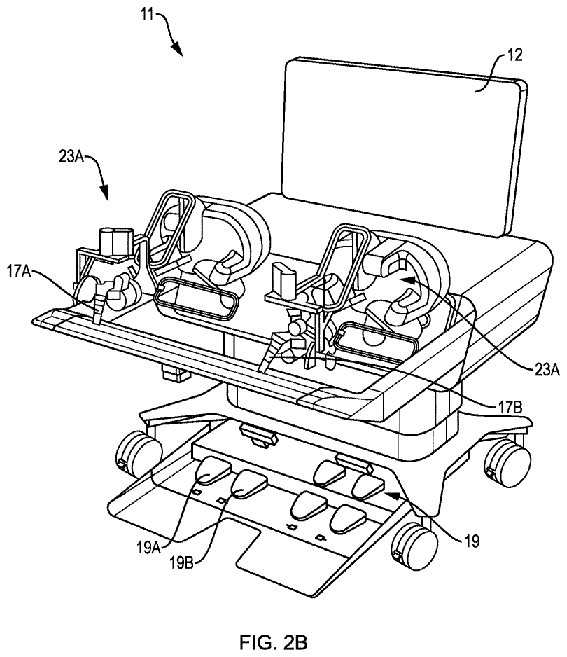

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. Robotic arm assembly 42 includes robotic arm 42A and robotic arm 42B. The subject 100 (e.g., a patient) is placed on an operation table 102 (e.g., a surgical table). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

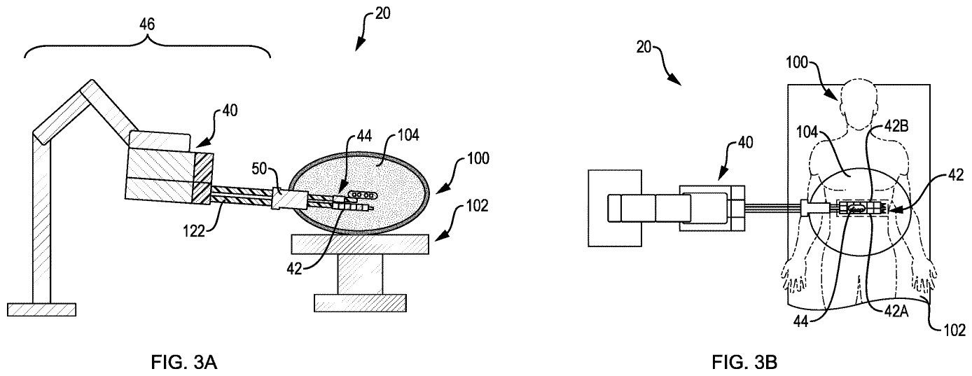

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

FIG. 5 illustrates a perspective front view of a portion of the robotic assembly 20 configured for insertion into an internal body cavity of a patient. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest.

In some embodiments, sensors in one or both of the first robotic arm 42A and the second robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, the camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Cooling a Robotic Camera

A cooling system for a robotic camera for a surgical robotic system as described herein can be employed with any of the surgical robotic systems described above or any other suitable surgical robotic system. Further, some embodiments described herein may be employed with semi-robotic endoscopic surgical systems that are only robotic in part.

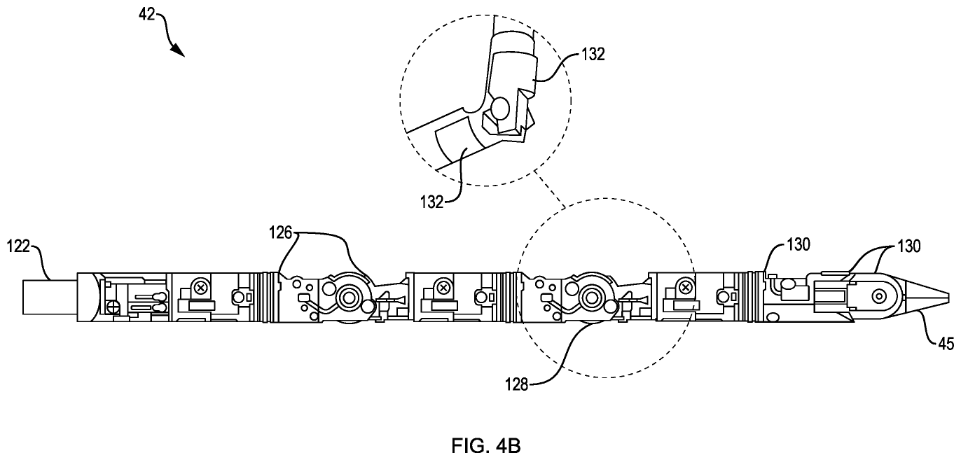

The cooling systems for a robotic camera may be understood with reference to embodiments depicted in FIGS. 6 to 17 and described below. For convenience, like reference numbers are used to referenced similar features of the various embodiments shown in the figures, unless otherwise noted.

FIG. 6 illustrates a schematic diagram of a camera and cooling system 200 including a camera housing 210 with a hollow chamber 225 and electronics components 213, a pump 256, and a reservoir 257. The camera housing 210 may also be referred to as a camera pill. A fluidic circuit 260 connects the pump 256, the hollow chamber 225, and the reservoir 257. The fluidic circuit includes segments or portions designated as a camera fluidic pathway 261A, 261A′, a transport fluidic pathway 261B, 261B′, an interconnection fluidic pathway 261C, 261C′, a reservoir fluidic pathway 261D, 261D′, and a pump to reservoir fluidic pathway 261E. The pump 256 establishes a fluid flow of fluid from the reservoir 257 through the fluidic circuit 260 to the hollow chamber 225 within the camera housing 210. Specifically, the pump 256 pressurizes a flow drawn from the reservoir 257 (via the reservoir fluidic pathway 261D′ and the reservoir fluidic pathway 261E) into the interconnection fluidic pathway 261C that continues sequentially into the transport fluidic pathway 261B, the camera fluidic pathway 261A, the hollow chamber 225, the camera fluidic pathway 261A′, the transport fluidic pathway 261B′, the interconnection fluidic pathway 261C′, the reservoir fluidic pathway 261D, and back to the reservoir 257. As fluid travels through the hollow chamber 225, the fluid absorbs heat generated by the electronics components 213 within the camera housing 210. The fluid then carries the heat out of the hollow chamber 225 and, accordingly, out of the camera housing 210 as the fluid returns to the reservoir 257. The fluid flow returns from the camera housing 210 to the reservoir 257 as a result of the pressure within the fluidic circuit 260 established by the pump 256.

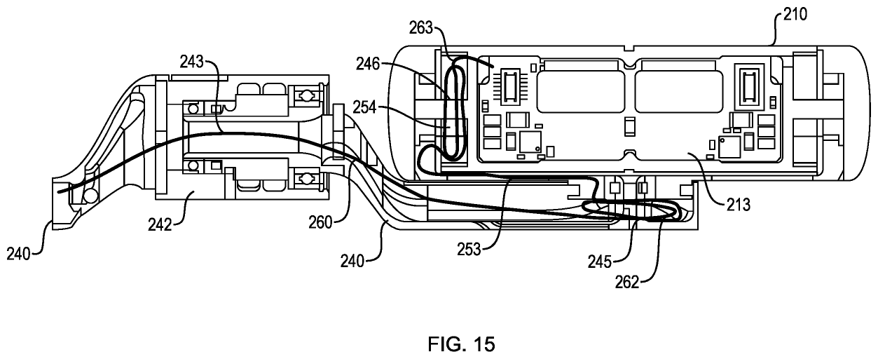

The reservoir 257 may permit heated portions of fluid to mix with portions of fluid at approximately the ambient temperature within the reservoir 257 and the contents of the reservoir may be passively cooled by exposure to the ambient environment of the reservoir 257. The reservoir 257 is positioned external to the patient. In some embodiments, the external location is exposed to ambient temperature so that the contents of the reservoir are maintained at approximately the room temperature in the return flow from the camera housing 210 is mixed with the contents of the reservoir 257. The reservoir 257 may be sufficiently large to act as a heat sink by absorbing heat from the fluid and transferring heat to the environment of the reservoir 257 to continue to provide cooling during the length of a surgical procedure and to maintain the temperature of the camera housing 210 within desired limits. In some embodiments, the external location is exposed to a chilled environment so that the contents of the reservoir are cooled to help facilitate heat removal from the camera housing 210. Other embodiments may be provided with a heat exchanger 370 (described below in connection with FIG. 16) to actively cool fluid and to assist in providing cooling during the length of a surgical procedure and to maintain the temperature of the camera housing 210 within desired limits. For example, the temperature of the camera housing 210 may be maintained below a temperature that has been selected as optimal for operation of the electronic components 213, optimal for safety of a patient exposed to the camera housing 210, or both. Alternatively, the camera housing 210 may be maintained below a temperature determined to be a maximum operating temperature of the electronic components 213 or a maximum safe temperature for the safety of a patient exposed to the camera housing 210, or both.

The temperature may be established based in part on standards for safe temperatures for accessible parts of a medical device. For example, International Standard 60601-1 of the International Electrotechnical Commission (IEC), Table 23, provides that the maximum temperature for an external surface of an accessible part that is likely to be touched for a time “t”, for a metal surface, is 48 °C for t≥1 minute and Table 24 provides that the maximum temperature for an applied part having contact with a patient for a metal surface for 1 min≤t<10 minutes is 48 °C and for t≥10 minutes is 43 °C. Accordingly, it may be desirable to maintain a temperature of the camera housing 210 below 48 °C and preferably below 43 °C. It has been found that the temperature of the camera housing 210 without cooling may exceed 43 °C. It may also be appreciated that during a surgical procedure, for example, a laparoscopic procedure, the camera housing may be exposed to an ambient temperature within the body of a patient of approximately 37 °C. Because this temperature is relatively close to 43 °C, the transfer of heat from the camera housing to the surroundings without cooling is unlikely to be sufficient to maintain a temperature at or below 43 °C.

In some embodiments, a minimum desired temperature may be established, or the flow rate may be selected so as to provide an amount of cooling expected to approximately maintain a desired temperature, where the minimum desired temperature or the desired temperature is warm enough to prevent or control fogging or formation of condensation on or inside the camera housing 210. Additionally, in some embodiments, the camera may be turned on for a period of time before a procedure in order to warm to a desired temperature to control fogging.

As described herein, micro tubing having an inner diameter between about 0.1 mm and 0.5 mm, an outer diameter between about 0.75 mm and 1.5 mm, and a Shore A hardness of about 80 is suitable for use in one or more of the fluidic pathways forming the fluidic circuit 260 and particularly for use in the camera fluidic pathways 261A, 261A′. Micro tubing having a larger inner and outer diameter and a Shore A hardness of about 60 is also suitable for use in one or more of the fluidic pathways forming the fluidic circuit 260 and particularly for use in the transport fluidic pathways 261B, 261B′.

In some embodiments, the fluidic circuit 260 is formed from medical grade flexible tubing such as that offered by Saint-Gobain Performance Plastics of Malvern, PA under the trade name TYGON®. The tubing may be a medical grade polyvinyl chloride. Alternatively, the fluidic circuit 260 may be formed for a polymer independently selected from a group consisting of polyvinyl chloride, silicone, polyethylene, polyamide, polyurethane, and combinations thereof.

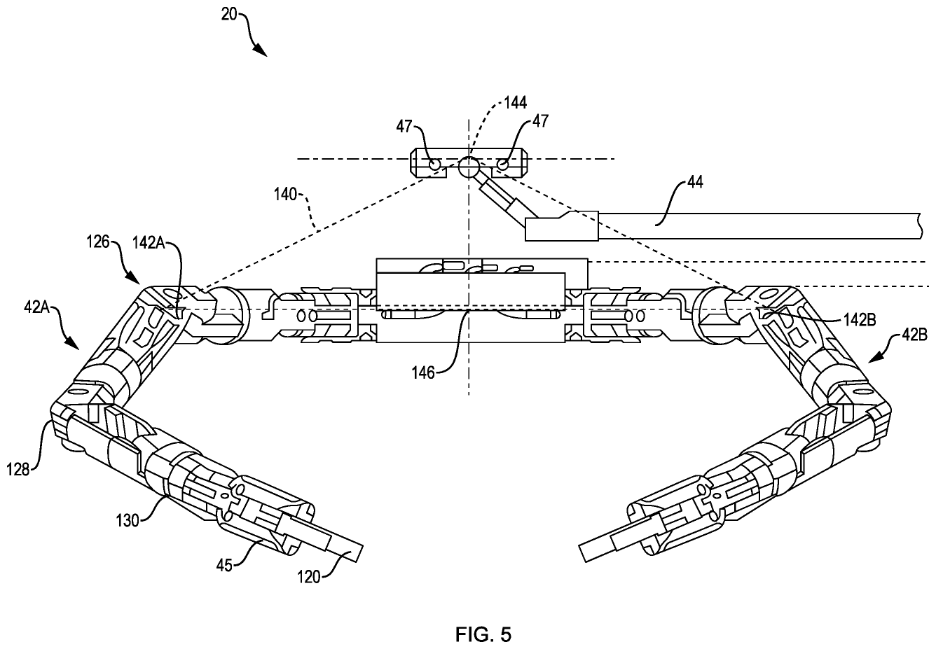

FIG. 7A depicts the camera assembly 205 in fluid communication with the camera and cooling system 200 in accordance with some embodiments including a camera housing 210 that is supported on a camera support assembly 240. The camera support assembly 240 is connected to a support arm 244. In some embodiments, the support arm 244 may have a tubular shape. In some embodiments, the support arm 244 may have an open profile. The support arm 244 connects to a mounting plate assembly 250. The mounting plate assembly 250 holds a housing 255 which may include the reservoir 257 for holding a cooling fluid and the pump 256 for pumping the cooling fluid. The camera assembly 205 is a fluid cooled camera assembly having a fluid cooling feature as described below suitable for use in place of the camera assembly 44. In some embodiments the mounting plate assembly 250 maybe situated at some distance from the camera housing 210 with the support arm 244 being provided at a suitable length.

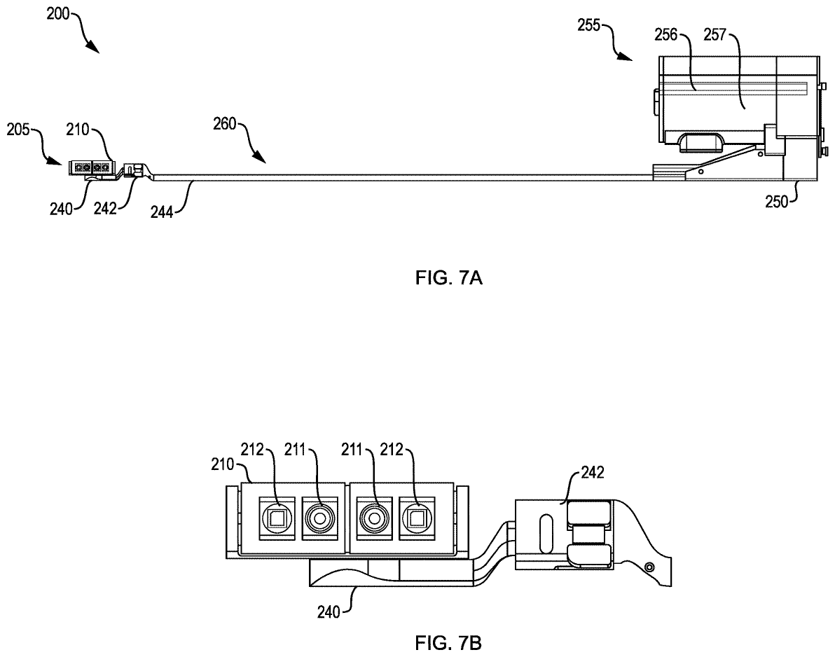

A camera fluidic circuit 260 may be established in the camera assembly 205 with the camera and cooling system 200 such that the pump 256 pressurizes a fluidic circuit that runs within or along the support arm 244 and the camera support assembly 240 to supply a fluid flow to the camera housing 210 and then return to the reservoir 257. The camera housing 210 may include a hollow chamber 225 (illustrated in FIG. 9) to cool the camera housing 210 using the fluid flow. The portion of the fluid flow within the camera housing 210 can receive heat from camera housing 210 and then remove the heat from the camera housing 210 as the fluid flows back to the reservoir 257. The portion of the fluidic circuit 260 at or near the reservoir 257 may be passively cooled by the ambient environment or may be actively cooled by a heat exchanger. In this way, the fluidic circuit 260 may cool the camera housing 210.

FIG. 7B illustrates in more detail the camera housing 210 housing one or more camera modules 211, and one or more light sources 212, a camera support assembly 240 and a roll axis joint 242. The light sources 212 may be light emitting diodes (LEDs). The roll axis joint 242 permits the camera housing 210 to move along the roll axis, i.e., to rotate about the long axis of the camera housing 210.

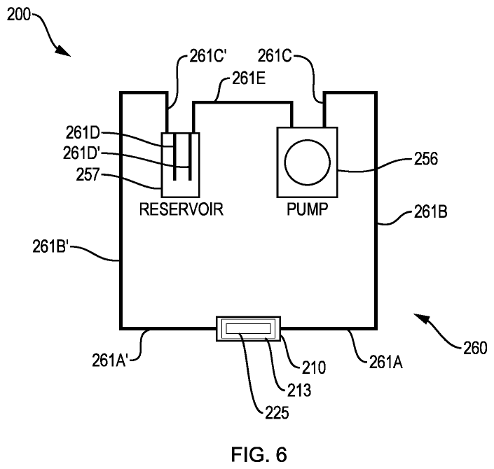

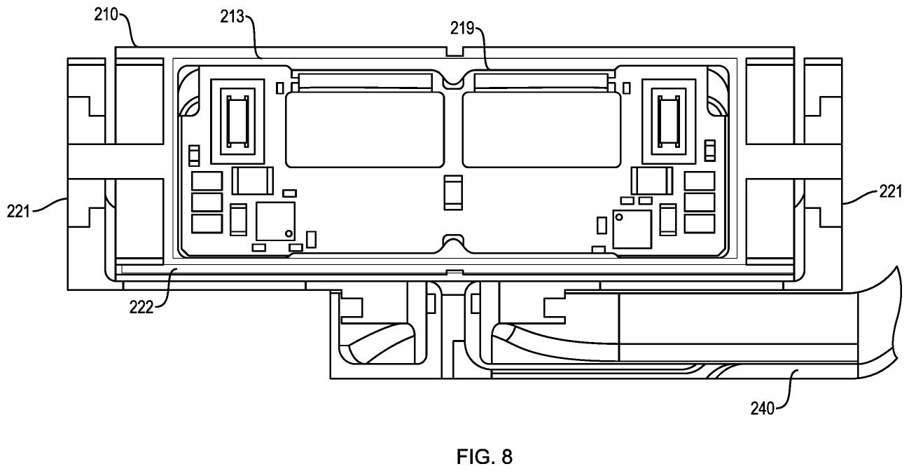

FIG. 8 illustrate a cross sectional view of the camera housing 210 along the long axis of the camera housing 210, looking from the backside of the camera housing 210 opposite the camera modules 211 and the light sources 212 and showing two bushings 221, an air gap 222, and a camera support assembly 240. As mentioned above, embodiments according to the present technology may mitigate the insulating effect of the air gap 222 and the bushings 221 wherein heat transfer out of the camera housing 210 is reduced. This cross sectional view of the camera housing 210 does not include the hollow chamber 225 or portions of the fluidic circuit 260 found in the camera housing 210. FIGS. 9, 10A and 10B illustrate the hollow chamber 225 and portions of the fluidic circuit 260 found in the camera housing 210. This cross sectional view of the camera housing 210 illustrates the portion of the interior of the camera housing 210 including the electronic components 213. The electronics components 213 may include the camera modules 211, the light sources 212, and other power consuming components (not shown) including one or more power supplies, one or more processors, one or more controllers, and one or more memory.

FIG. 9A illustrates a portion of the camera housing 210 with the electronic components 213 removed in order to illustrate that in some embodiments the hollow chamber 225 is formed along an interior wall 214 of the camera housing 210, along with the ingress aperture 226, and the egress aperture 227. Those skilled in the art will appreciate that location of the ingress aperture 226, and the egress aperture 227 is based on the flow direction of the fluid flowing into and out of the hollow chamber 225.

FIG. 9B illustrates components in the interior of the camera housing 210. In some embodiments, the camera housing 210 may be assembled from a two-part clam shell construction with the two pieces connected, e.g., by laser welding. In some embodiments, the camera housing 210 may be a single-piece design. A plate 229 is shown placed over and sealing the hollow chamber 225.

FIG. 10A illustrates the hollow chamber 225 with the plate 229 introduced over the hollow chamber 225. The hollow chamber 225 is formed along an interior wall 214 of camera housing 210. An arrow 230A illustrates the direction of fluid flow entering the hollow chamber 225 at the ingress aperture 226 at a first temperature. An arrow 230B illustrates fluid flow through the hollow chamber 225. An arrow 230C indicates fluid flow exiting the hollow chamber 225 at the egress aperture 227 at a second temperature that is higher than the first temperature, after the fluid has absorbed height from the camera housing 210 while within the hollow chamber 225. In some embodiments, the hollow chamber 225 may permit flow to be largely uncontrolled within the hollow chamber 225. In other embodiments, structural features may be imposed in order to establish certain fluid flow paths through the hollow chamber 225. FIG. 10B illustrates the opposite portion of the camera housing 210 including the camera modules 211, the light sources 212 and the electronic components 213. The hollow chamber 225 is formed along the interior wall 214 of camera housing 210 that is opposite to the camera modules 211 when camera housing 210 is assembled. When the camera housing 210 is assembled, the hollow chamber 225 is proximate the electronic components 213 within the camera housing 210. The spaces surrounding the electronic components 213 may be filled with a thermally conductive material 232, for example a thermally conductive putty, to facilitate heat transfer from the camera housing 210 and the electronic components 213 to the fluid flowing through the hollow chamber 225.

The plate 229 may separate the various electronic components 213 from the hollow chamber 225 such that the electronics components 213 do not come in direct contact with fluid from the hollow chamber 225. The plate 229 may be connected to the camera housing 210 by laser welding. Alternatively, the plate 229 may be connected to the camera housing 210 by another means, such as an adhesive or solder, or may be held in place by structure of the camera housing 210, such as a slot or groove. In some embodiments, the hollow chamber 225 has a shape and a volume defined by a geometry of the camera housing 210 and a geometry of the plate 229. For example, the hollow chamber 225 has a shape and a volume defined by an interior wall 214 of the camera housing 210 and a wall of the plate 229 as well as end walls 218 define the hollow chamber 225.

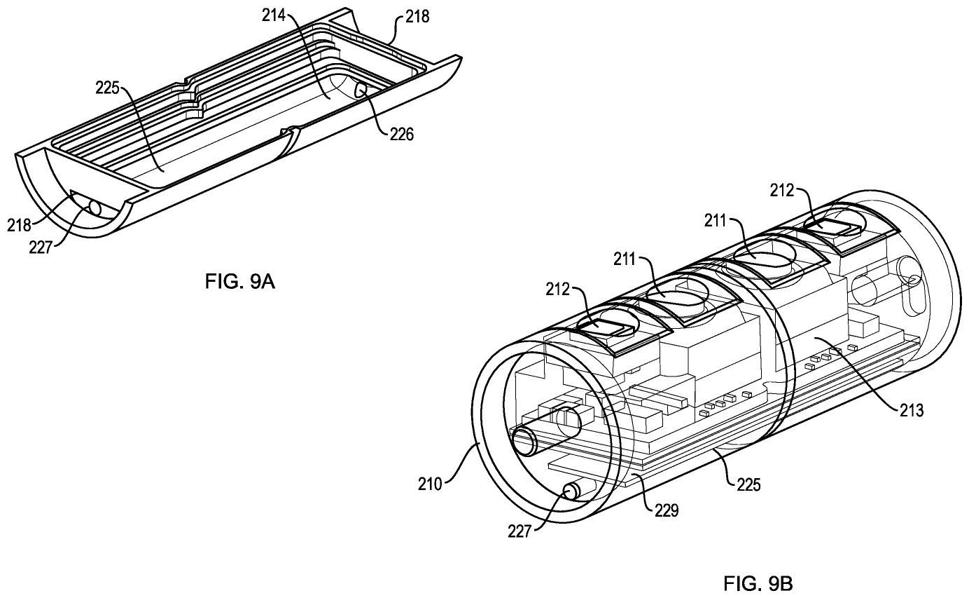

FIG. 11 illustrates a perspective view of a camera assembly 205 including camera housing 210 that is partially transparent to illustrate heat absorption by the fluid flowing through the hollow chamber 225. The hollow chamber 225 includes an ingress aperture 226 and an egress aperture 227. As maybe observed by comparing FIG. 8 and FIG. 11, the hollow chamber 225 is positioned proximate to the side of the electronics components 213, which is opposite to the camera modules 211 and the light sources 212. Heat generated by the electronic components 213 (including the camera modules 211 and the light sources 212 and any other power consuming component) may be absorbed into the hollow chamber 225 and into fluid flowing through the hollow chamber 225. In some embodiments, during operation of the camera assembly 205, a fluid is introduced into the hollow chamber 225 at the ingress aperture 226 and permitted to flow through the hollow chamber 225, where heat generated by the electronic components 213 is absorbed. In turn, the thermodynamic properties of the fluid cools the electronic components 213 and in turn the whole camera assembly 205 and transports the heated fluid outside of the camera housing 210 via the egress aperture 227. In some embodiments the direction of the fluid flow maybe reversed.

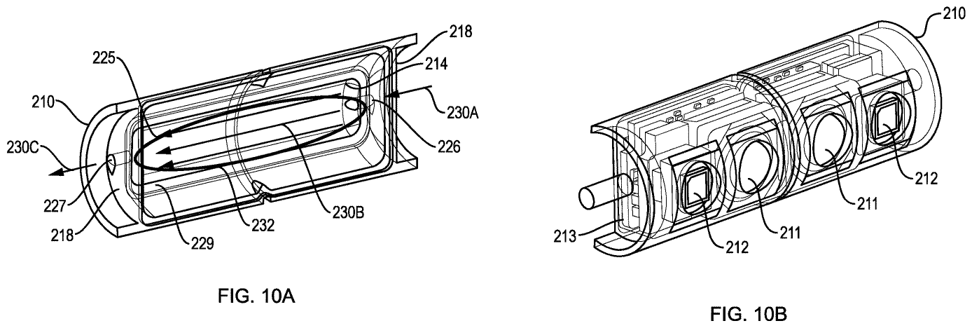

FIG. 12 illustrates a cross sectional view of the camera housing 210 taken along an axis transverse to the long axis of the camera housing 225 showing the hollow chamber 225 enclosed by the plate 229 and separated from the electronic components 213. One of the apertures (ingress aperture 226 or egress aperture 227) is shown providing access to the hollow chamber 225.

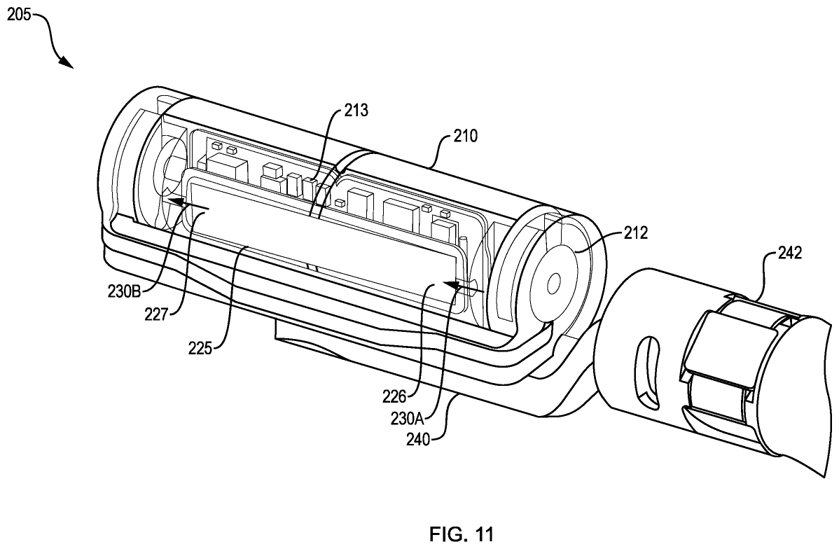

FIG. 13 illustrates the camera housing 210, the camera support assembly 240, and the roll axis joint 242. Within the camera support assembly 240, a channel 253 is formed. The channel 253 may receive tubing (such the segments of the fluidic circuit 260 illustrated in FIG. 6) to carry fluid within the fluidic circuit 260.

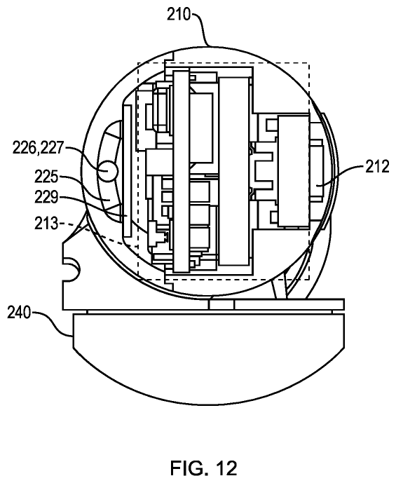

FIG. 14 illustrates the camera housing 210 and the camera support assembly 240 additionally provided with a pitch activation cable 351, a yaw drive cable 355, and a portion of fluidic circuit 260 formed from micro tubing. An example of suitable micro tubing is identified above. The portion of fluidic circuit 260 is positioned at least partially within the channel 253 and within the gaps 254 inside of the bushings 221 on either side of the camera housing 210. The fluidic circuit 260 may be threaded into the ingress aperture 226 and the egress aperture 227 of the hollow chamber 225 (not shown).

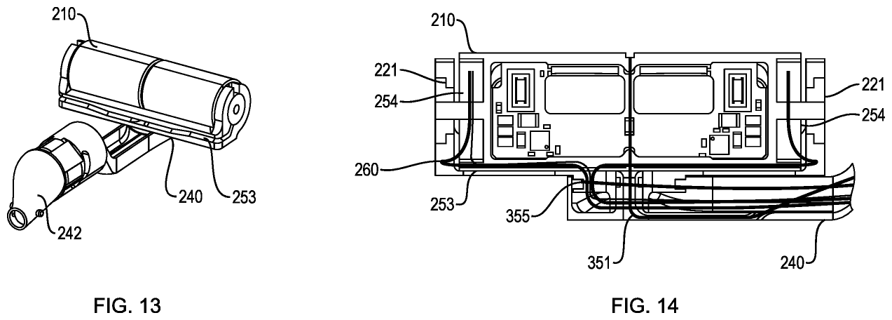

FIG. 15 illustrates the fluidic circuit 260 introduced from the support arm 240 and passing through the interior of roll axis joint 242 and along camera support assembly 240 into the yaw axis joint 245. The fluidic circuit 260 passes through an open channel 243 through the roll axis joint 242, accordingly fluidic circuit 260 is not affected by rotation of the roll axis joint 242. The fluidic circuit 260 includes a first plurality of coils 262 at the yaw axis joint 245 in order to permit rotation of the camera housing 210 about the yaw axis joint 245 without constraint by the fluidic circuit 260. In an embodiment, the first plurality of coils 262 may permit 540 degrees of rotation in the yaw axis joint 245. The fluidic circuit 260 then continues through part of a channel 253 to a gap 254 surrounding the pitch axis joint 246. At the pitch axis joint 246, the fluidic circuit 260 includes a second plurality of coils 263 to permit rotation of the camera housing 210 about the pitch axis joint 246 without constraint by the fluidic circuit 260. In an embodiment, the second plurality of coils 263 may permit 330 degrees of rotation in the pitch axis joint 246. Thereafter, the fluidic circuit 260 passes into camera housing 210 where it connects with the hollow chamber 225 (not shown) to extract heat generated within the camera housing 210 by electronic components 213.

FIG. 16 illustrates the camera and cooling system 301 featuring a pump 256. In some embodiments, the pump 256 is a peristaltic pump. In some embodiments, the pump 256 is driven by a pump motor 241. By separating the pump motor 241 from the pump 256, it may be possible to treat the pump 256 as disposable while reusing the pump motor 241. The pump 256 may also be sterilized more readily. The camera and cooling system 301 also includes the fluidic circuit 260. The fluidic circuit 260 can also include a fill port 360 and a purge valve 365 that may be used to fill the reservoir 257 and the remaining components of the system. In some embodiments, the fluidic circuit 260 may be provided pre-filled with a fluid. In some embodiments, the fluidic circuit 260 may be filled on-site before a procedure. The fluidic circuit 260 additionally connects the reservoir 257 with bent tubing 228 within the hollow chamber 225. In some embodiments, providing bent tubing 228 within the hollow chamber 225 may improve fluid flow through hollow chamber 225 by reducing low-flow zones that may occur in embodiments of the hollow chamber without channels. In some embodiments, the fluidic circuit 260 also connects to a heat exchanger 370. The heat exchanger 370 may be provided to actively cool fluid within the fluidic circuit, thereby increasing the cooling capacity available.

In operation, the pump 256 may be operated to drive fluid past the fill port 360 to the reservoir 257 to pressurize the reservoir 257 and initiate a flow of relatively cool fluid past the purge valve 365, along the camera support assembly 240 to the hollow chamber 225. At this stage, the fluid absorbs heat from the electronics components 213 housed in the camera housing 210 and then travels to the optional heat exchanger 370, which may be used to actively cool the fluid before it returns to the pump 256 and the reservoir 257. In embodiments without a heat exchanger, the reservoir and portions of the tubing at room temperature may provide sufficient cooling of the fluid for operation of the cooling system. This process and fluid flowing in the fluidic circuit 260 cools the camera housing 210. For example, the fluidic circuit 260 is able to cool the camera housing 210 to maintain a temperature below 48 °C and preferably below 43 °C.

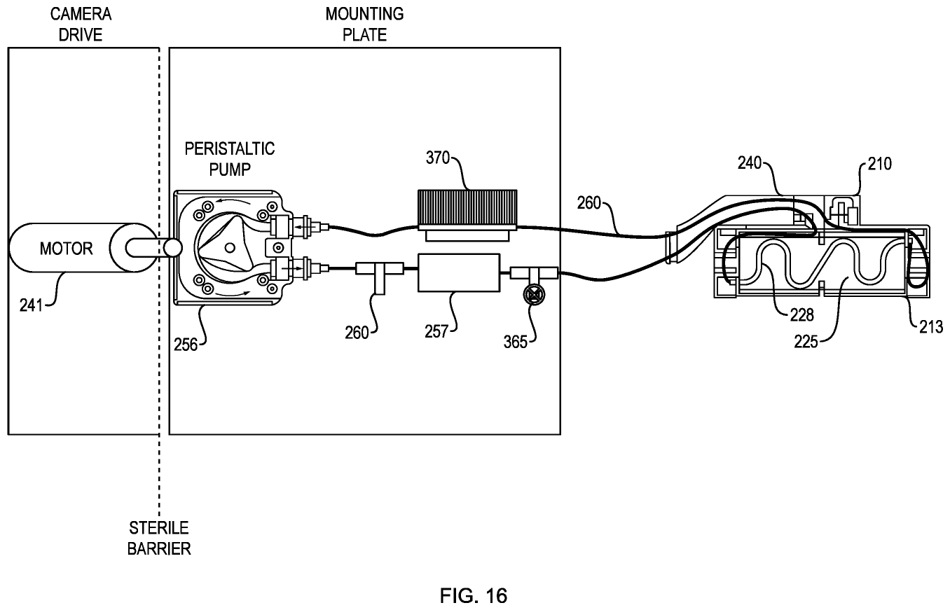

FIG. 17A illustrates a cross sectional view of a camera housing in accordance with some embodiments taken along plane A as shown on FIG. 17B. FIG. 17A illustrates the hollow chamber 225 enclosed on one side by the plate 229. Also seen in relationship to the hollow chamber 225 are the camera module 211, the electronic components 213, and the thermally conductive material 232. One of the apertures (ingress aperture 226 or egress aperture 227) is shown providing fluid access to the hollow chamber 225. An arrow 290 indicates transmission of heat generated by the electronic components 213 to the hollow chamber 225 through, at least in part, the thermally conductive material 232. The thermally conductive material 232 has a shape and position to abut and receive heat from the electronic components 213 and to abut and transmit heat to the plate 229 and the hollow chamber 225.

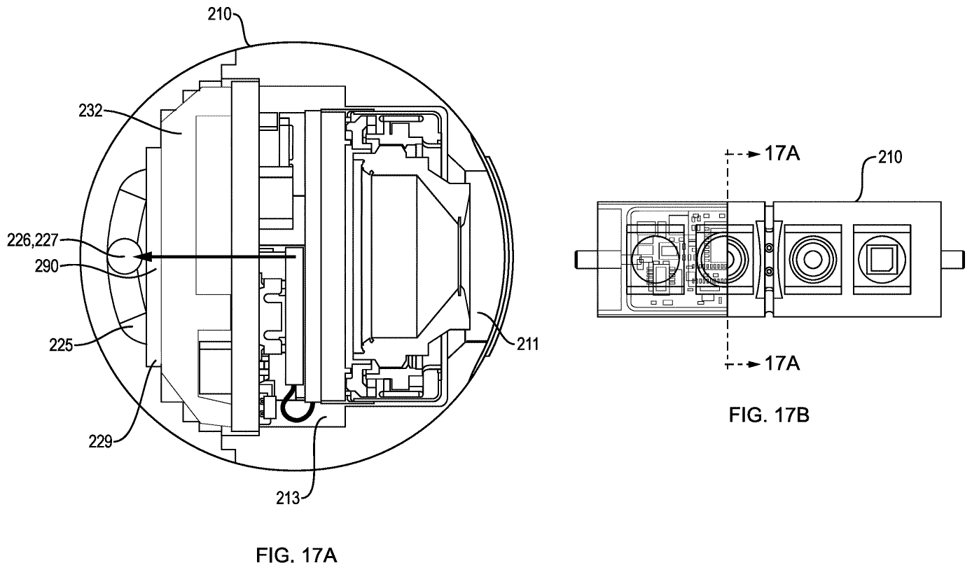

FIG. 18 illustrates a portion of the camera housing 210 including the hollow chamber 225 formed along an interior wall 214 of the camera housing 210. In some embodiments, the hollow chamber 225 includes a series of channels 231. The channels 231 may improve fluid flow through the hollow chamber 225 increasing the flow rate through the hollow chamber 225 and further increasing cooling capacity. The channels 231 may improve distribution of fluid through the hollow chamber 225, facilitating heat conducting into the fluid and increasing the consistency of the cooling.

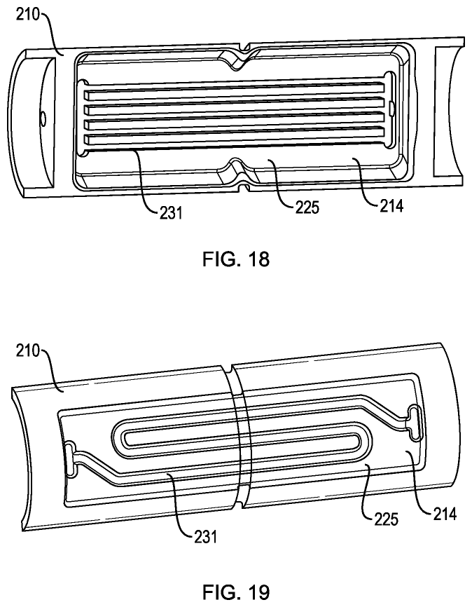

FIG. 19 illustrates a portion of the camera housing 210 including the hollow chamber 225 formed along an interior wall 214 of the camera housing 210. In some embodiments, the hollow chamber 225 includes a channel 231 that is inset along a portion of the interior wall 214 of the camera housing 210. For example, the channel 231 may be etched or otherwise formed into the interior wall 214, for example, by molding or machining or another suitable technique. In the embodiment shown in FIG. 19, a cover (not shown) may be provided over the follow chamber 225 forming an outside wall of the camera housing 210.

### Example Calculation

In some embodiments, a fluid flow rate of 5 mL/min or more is established in the fluidic circuit 260 to provide cooling to the electronic components 213 housed in the camera housing 210. For example, where the camera fluidic circuit 260 (such as the fluidic pathways 261A, 261A′) has a length of about 15 cm (including coils) and an internal diameter of about 0.25 mm and the transport fluidic pathway (such as the transport fluidic pathways 261B, 261B′) has a length of about 1 m and an internal diameter of about 0.75 mm, it has been found that a pressure of approximately 30 psi at the pump can achieve a flow rate of about 5 mL/min.

In order to confirm the sufficiency of a particular flow rate, such as 5 mL/min, for in a camera housing, a calculation may be performed to determine the change in temperature within the fluid at a particular flow rate to remove the heat expected to be generated by the electronic components 213. In order to determine an adequate fluid flow rate, such as, for example, the fluid flow rate of 5 mL/min, a calculation may be performed to determine the flow needed to remove the heat emitted by the electronic components. For example, in an embodiment, the amount of power dissipated by the electronics components 213 has been calculated to be 2 W. The fluid flow needed may be determined according to Eq. 1:

$$Δ T = \frac{W}{c dF} \qquad ( Eq . 1 )$$

where F is the fluid flow, W is the power to be dissipated from the electronic components 213, c is the specific heat of the fluid, d is the density of the fluid, and ΔT is the difference in temperature entering and exiting the hollow chamber. For example, where the power to be dissipated is 2 W, the fluid is water with a specific heat of 4.184 J/g °C, F is 5.0 ml/min, calculated as follows:

$$Δ T = \frac{W}{cdF} = ( \frac{2 W}{( 4.1 \; 84 \frac{J}{g °C} ) ( \frac{1 g}{1 ml} ) ( 5. ml / min )} ) ( \frac{1 J / s}{1 W} ) ( \frac{60 s}{1 min} ) = 5.74 °C$$

The resulting ΔT of 5.74 °C represents the increase in the water temperature necessary to receive the heat generated if up to 2 W is dissipated by the electronics components as heat.

What is claimed is:

1. A robotic surgical system comprising:
a housing including at least one camera module, one or more electronic components, and a hollow chamber proximate to the one or more electronic components;
a camera support assembly supporting the housing and including an arm with at least a first joint;
a pump;
a fluidic circuit in liquid communication with the hollow chamber and the pump and extending along at least part of the camera support assembly.

2. The robotic surgical system of claim 1, in which the hollow chamber is formed along an interior wall of the housing opposite the at least one camera module and includes an ingress aperture and an egress aperture.

3. The robotic surgical system of claim 1, further comprising a first plate in the housing forming a wall of the hollow chamber.

4. The robotic surgical system of claim 2, wherein the hollow chamber comprises a plurality of liquid channels therein.

5. The robotic surgical system of claim 1, further comprising a thermally-conductive material contacting at least a portion of the at least one camera module, the one or more electronic components, and a wall of the hollow chamber.

6. The robotic surgical system of claim 1, wherein the fluidic circuit includes a first portion having a first inner diameter and a first outer diameter and a second portion having a second inner diameter and a second outer diameter.

7. The robotic surgical system of claim 6, wherein the second inner diameter is greater than the first inner diameter.

8. The robotic surgical system of claim 6, wherein the first inner diameter is between about 0.1 mm and about 0.5 mm and the first outer diameter is between about 0.75 mm and about 1.5 mm.

9. The robotic surgical system of claim 6, wherein the first inner diameter is about 0.25 mm and the first outer diameter is about 0.75 mm.

10. The robotic surgical system of claim 6, wherein the second inner diameter is between 0.5 mm and 1 mm.

11. The robotic surgical system of claim 6, wherein the second inner diameter is about 0.75 mm.

12. The robotic surgical system of claim 6, wherein the second inner diameter is approximately equal to the first outer diameter.

13. The robotic surgical system of claim 6, wherein at least the first portion of the fluidic circuit is a kink-resistant polymer tubing.

14. The robotic surgical system of claim 6, wherein the first portion and the second portion of the fluidic circuit comprise a polymer independently selected from a group consisting of polyvinyl chloride, silicone, polyethylene, polyamide, polyurethane, and combinations thereof.

15. The robotic surgical system of claim 6, wherein the first portion and the second portion of the fluidic circuit comprise medical grade polyvinyl chloride.

16. The robotic surgical system of claim 6, wherein at least the first portion of the fluidic circuit comprises a polymer material with a Durometer Shore A hardness rating of about 80.

17. The robotic surgical system of claim 1, further comprising a fill port and a purge valve in liquid communication with the fluidic circuit.

18. The robotic surgical system of claim 1, further comprising an external reservoir in liquid communication with the fluidic circuit.

19. The robotic surgical system of claim 1, wherein the fluidic circuit comprises a first coil of tubing proximate the first joint.

20. The robotic surgical system of claim 1, wherein the camera support additionally comprises a second joint and the fluidic circuit comprise a second coil of tubing proximate the second joint.

21. The robotic surgical system of claim 1, wherein the first coil of tubing comprises a number of coils sufficient to permit 540 degrees of rotation by the first joint.

22. The robotic surgical system of claim 1, wherein the pump is a peristaltic pump.

23. The robotic surgical system of claim 1, wherein the pump is configured to be driven by a pump motor.

24. The robotic surgical system of claim 1, wherein the pump comprises a plurality of sets of rollers.

25. The robotic surgical system of claim 1, wherein the robotic surgical system has an internal portion and an external portion, the internal portion including the housing and the camera support and being configured to be placed at least partially inside a patient during a surgical procedure, and the external portion configured to be outside a patient during a surgical procedure.

26. The robotic surgical system of claim 25, wherein the pump is in the external portion.

27. The robotic surgical system of claim 1, further comprising an external reservoir in liquid communication with the fluidic circuit.

28. The robotic surgical system of claim 1, wherein the housing and the camera support form at least part of a laparoscopic camera.

29. A method for cooling a robotic surgical system, the method comprising
providing a robotic surgical system including a housing with at least one camera module, one or more electronic components, and a hollow chamber proximate to the one or more electronic components; and
pumping a liquid from an external reservoir through a fluidic circuit including the hollow chamber.

30. The method of claim 29, further comprising removing heat from the hollow chamber of the housing by means of the liquid.

31. The method of claim 30, wherein removing heat from the hollow chamber removes sufficient heat to maintain a temperature at or below about 43 °C on an exterior surface of the electronic housing proximate the hollow chamber.

32. The method of claim 29, wherein pumping a liquid through a fluidic circuit including the hollow chamber and an external reservoir comprises establishing a liquid flow sufficient to maintain a temperature within the housing of about 70 °C or less.

33. The method of claim 29, wherein pumping a liquid through a fluidic circuit including the hollow chamber and an external reservoir comprises establishing a liquid flow of about 5 mL/minute or more.

34. The method of claim 29, wherein pumping a liquid through a fluidic circuit including the hollow chamber and an external reservoir comprises pumping at a pressure of 30 psi or greater.

35. The method of claim 29, further comprising permitting a portion of the liquid to cool passively in the reservoir.

36. The method of claim 29, further comprising actively cooling a portion of the liquid using a heat exchanger.

37. The method of claim 29, further comprising rotating a joint of the robotic surgical system while permitting coiling or uncoiling of a tube coil proximate to the joint.

38. The method of claim 29, wherein the liquid is selected from distilled water or saline.

39. A minimally invasive surgical camera comprising,
a housing;
one or more cameras accommodated within the housing;
one or more circuit board assemblies provided within the housing;
a hollow cavity in proximity to the one or more circuit board assemblies, the hollow cavity including a liquid ingress port, a liquid egress port and an interior surface defined by the housing.

40. The minimally invasive surgical camera of claim 39, further comprising a first plate within the housing forming a wall of the hollow cavity.

41. The minimally invasive surgical camera of claim 39, wherein the liquid ingress port and the liquid egress port are configured to receive a polymer tube.

42. The minimally invasive surgical camera of claim 39, wherein the hollow cavity comprises a plurality of liquid channels.

43. The minimally invasive surgical camera of claim 42, wherein the plurality of liquid channels are formed by machining channels in the interior surface, the surface of the first plate, or both.

44. The minimally invasive surgical camera of claim 39, further comprising a thermally-conductive material within the housing and contacting at least a portion of the one or more cameras, the one or more circuit board assemblies, and the hollow cavity.

45. The minimally invasive surgical camera of claim 39, wherein the hollow cavity is configured to receive a liquid flow at the liquid ingress port and to release the liquid flow at the liquid egress port.

46. The minimally invasive surgical camera of claim 45, wherein the liquid flow in the hollow cavity receives heat emitted by the one or more cameras, the one or more circuit board assemblies, or both.

47. The minimally invasive surgical camera of claim 45, wherein the liquid flow removes heat from the hollow cavity.
